(19) United States
(12) Patent Application Publication
(10) Pub. No.: US 2024/0299762 A1
(43) Pub. Date: Sep. 12, 2024

(54) ENERGIZER

(71) Applicants: Ling-Ho LAM, San Jose, CA (US); Raymond Yim CHIU, San Jose, CA (US)

(72) Inventors: Ling-Ho LAM, San Jose, CA (US); Raymond Yim CHIU, San Jose, CA (US)

(21) Appl. No.: 18/405,466

(22) Filed: Jan. 5, 2024

(51) Int. Cl. A61N 2/00; A61N 2/02; A61N 5/06

(52) U.S. Cl. CPC A61N 2/002; A61N 2/02; A61N 5/06; A61N 2005/0626; A61N 2005/0652; A61N 2005/0659; A61N 2005/0663; A61N 2005/1089

(57) ABSTRACT

An energizer that includes a plurality of energy producing modules each configured to emit a respective energy output of a type different from energy outputs of other modules, and an energy field integrator arranged to receive the energy outputs of the energy producing modules, to integrate the received energy outputs, and to output an integrated output, the energy field integrator comprising a plurality of gemstones arranged in an ordered array.

### FIELD OF THE INVENTION

The present invention relates to an energizer device that generates a torsion field by integrating multiple types of energy.

### BACKGROUND OF THE INVENTION

Nearly every medical and healing system has a concept of energy. The focus of conventional western medicine is the biochemical energy that enables cell functions mediated by ATP (adenosine triphosphate). Nearly all other traditional fields of medicine acknowledge an invisible healing force or life energy. The life energy is called prana in the Ayurvedic tradition of yoga, qi in traditional Chinese edicine (TCM), pnetuna in classical Greece, mach in Jewish mysticism, and in western vitalism, animal magnetism, elan vital, orgone, and “charge”. It is believed that the free flow of life energy confers health and vitality. Life energy, it is believed, can return to balance from a state of imbalance caused, for example, by an outside force such as stress. It is also believed that the life energy is in spinning motion and if the spinning slows down due to, for example, illness, the spinning motion may not be recovered to a healthy rate. But if the spinning slows down (i.e., illness), the gyroscope won't be able to recover; it will fall over and die. Energy medicine (EM) is a set of techniques designed to keep the life energy spinning.

Biofield model encompasses the invisible life energies. According to the National Institute of Health (NIH) the biofield is “a massless field, not necessarily electromagnetic, that surrounds and permeates living bodies and affects the body”, which are thought to be endogenously generated and are essential to understanding EM. See The field of energy medicine (EM) extracted from https://pubmed.ncbi.nlm.nih.gov/33168457.

Biophotons and torsion fields serve as a bridge between the twentieth century view of an organism as a chemical machine and the new view of life as communications and energetic flows, in which DNA is the source of biophotons, governing cellular functionality, communications and behavior, and growth and metabolism, with high energetic efficiency. The coherent biophoton field forms a hologram throughout the body, informing each DNA molecule where it fits in the overall blueprint, and governing cell differentiation and specialization.

A torsion wave is created with the creation of each biophoton. A torsion wave is a widespread and important form of radiation that couples particle spins together, and propagates through space as a twisting wave in the metric, causing a variety of new effects that have been studied extensively for the past forty years, at multiple research centers in Russia, which have concluded that the torsion force is exactly the same as subtle energy.

Torsion behavior can be predicted mathematically, and inventions and practical devices have been developed using torsion waves. While the biophotons carry electromagnetic energy and are contained mostly within the body, torsion waves easily penetrate tissue and form the primary holographic pattern outside the body, including the subtle structure of the aura and chakras, and also the long range signals used in distance energy healing.

It has been shown that the torsion field is coupled to consciousness, and is a higher dimensional field with additional degrees of freedom that are not found in Western science. Torsion fields obey Yang-Mills gauge equations, can alter the rate of time and produce stable energy forms without the need for matter. Torsion fields may explain many kinds of “spiritual” phenomena that mainstream physics ignores. Torsion fields appear to be what is needed to develop a true science of consciousness and understand the nature of life.

### SUMMARY OF THE INVENTION

It is an object of the present invention to provide a device that integrates the energies of, for example, visible light, free electrons, a magnetic field, infrared, and low level light to boost toxin removal and recovery of the damaged brain cells and neural networks.

An energizer according to the present invention includes a plurality of energy producing modules each configured to emit a respective energy output of a type different from energy outputs of other modules; and an energy field integrator arranged to receive the energy outputs of the energy producing modules, to integrate the received energy outputs, and to output an integrated output, the energy field integrator comprising a plurality of gemstones arranged in an ordered array.

The integrated output may be a torsion field, for example, a spherical torsion field.

The plurality of gemstones may include spherical gemstones and cubical gemstones.

The energy field integrator may include a base panel, and at least one output plate, wherein the base panel is at least partially transparent to the outputs of the modules, and wherein the gemstones reside between the base panel and the at least one output plate.

The at least one output plate may include a plurality of spaced perforations arranged to create wave diffraction patterns.

The energy field integrator may include a tray having a plurality of receptacles that receive the plurality of gemstones. The tray may be at least partially transparent to the outputs of the modules.

The modules include at least a visible light generator module, a magnetic field generator module, an infrared generator module, and a free electron generator module.

The energizer may include a microcontroller that controls the modules.

The energizer may further include a body infrared signal generator module.

The visible light generator module includes a plurality of LED components serving as output components thereof that, for example, emit light with wavelengths of 650 nm, 525 nm, 380 nm, and 590 nm.

The infrared generator module may have an infrared (IR) generating coil serving as its output component that emits infrared in the range 6000 nm to 12000 nm.

The magnetic field generator module may have an electromagnet serving as its output component and generates a magnetic field in the range 0.1 to 80 microteslas.

The free electron generator module is a DC power generator that generates, for example, a static electric field of 850 V.

The infrared generator module and the visible light generator module may be configured to be cyclically operated at a frequency set by a user. The frequency may be 7.83 Hz or a harmonic thereof.

The energizer may be configured to permit the outputs of the modules to be adjusted in response to user instructions. The energizer may be configured to receive user instructions from a user-operable mobile device.

Other features and advantages of the present invention will become apparent from the following description of the invention which refers to the accompanying drawings.

### BRIEF DESCRIPTION OF THE DRAWINGS

FIG. 1 is a schematic illustration of an energizer according to the present invention.

FIG. 2 shows a circuit diagram of the electronic parts of an energizer according to the present invention.

FIG. 3A shows a top perspective view of a physical embodiment of an energizer according to the present invention.

FIG. 3B shows a bottom perspective view the physical embodiment seen in FIG. 3A.

FIG. 3C is the physical embodiment of FIGS. 3A and 3B in a disassembled state.

FIG. 4A shows the gemstones of the energy field integrator arranged in a tray.

FIG. 4B shows the tray of FIG. 4A with some receptacles empty and other receptacles filled with gemstones.

FIG. 5A shows led components arranged on the circuit board.

FIG. 5B shows the permanent magnet component of the energizer according to the present invention.

FIG. 5C shows an electromagnet used in the magnetic generator module.

### DETAILED DESCRIPTION OF EMBODIMENTS OF THE INVENTION

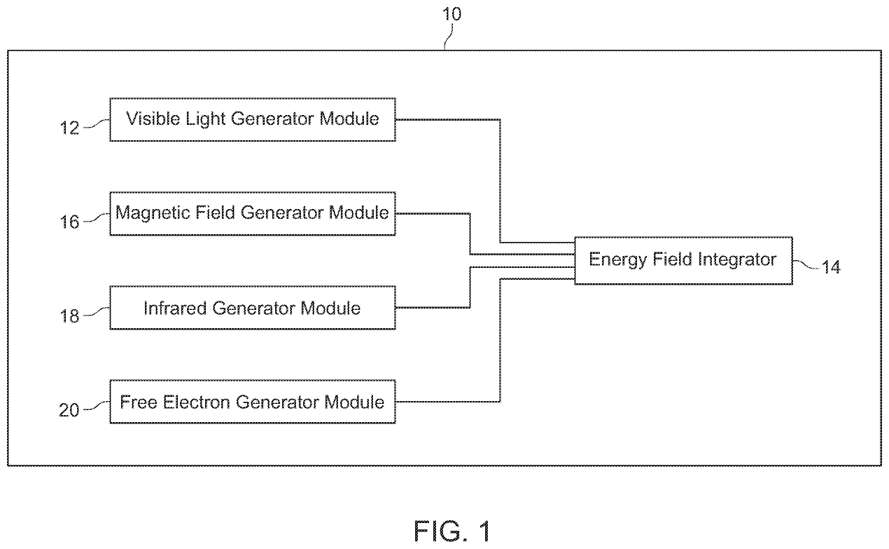

Referring to FIG. 1, an energizer according to the present invention integrates a number of energy sources to output an integrated energy field that reinforces the weakened energy field of the body to boost the vitality of brain cells and neural networks.

An energizer 10 according to the present invention may include a visible light generator module 12, a magnetic field generator module 16, an infrared generator module 18, and a free electron generator module 20. The outputs of the visible light generator module 12, the magnetic field generator module 16, the infrared generator module 18, and the free electron generator module 20 are received by an energy field integrator 14. The energy field integrator 14 integrates the outputs of the visible light generator module 12, the torsion field generator module 14, the magnetic field generator module 16, the infrared generator module 18, and the free electron generator module 20 and outputs the integrated energy field.

The energy field integrator 14 includes a plurality of polished gems as further described below that function as a body infrared generator, and example of which is disclosed in U.S. Ser. No. 18/485,389, filed Oct. 12, 2023, the disclosure of which is incorporated by this reference.

The visible light generator module 12 may generate multiple (for example, four) wavelengths (colors) of light.

The magnetic field generator module 16 generates a magnetic field in the range 0.1-80 microteslas.

The infrared generator module 18 generates electromagnetic waves with wavelengths in the range 6000 nm to 12000 nm.

The free electron generator module 20 is a DC power generator.

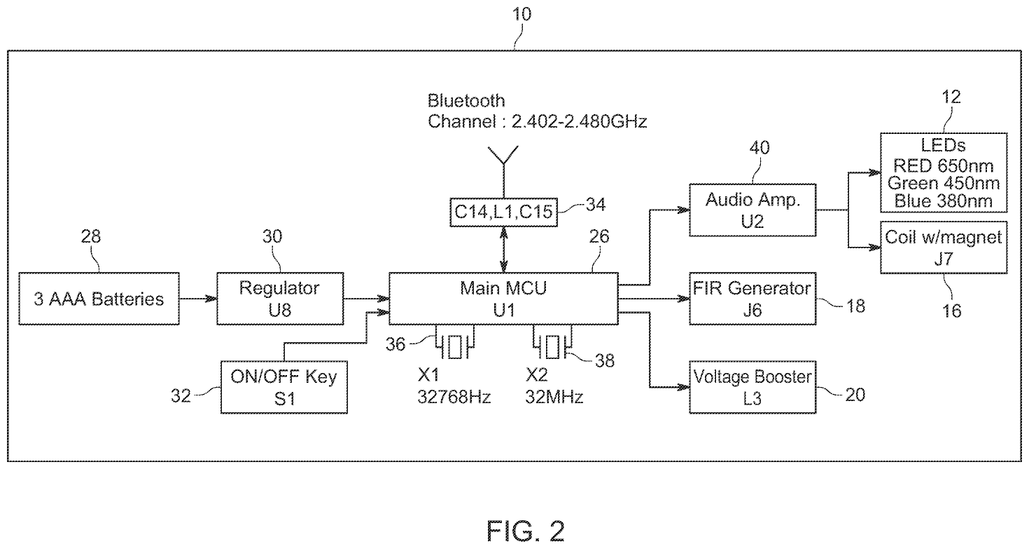

Referring to FIG. 2, an energizer 10 according to the present invention may have a main (microcontroller unit) MCU 26 that operates the visible light generator module 12, the magnetic field generator module 16, the infrared generator module 18, and the free electron generator module 20. The energizer 10 is preferably powered by a battery 28 or a plurality of batteries 28, which may be rechargeable. Alternatively, an energizer 10 according to the present invention may be powered by an AC to DC converter that receives line power.

A power regulator 30 may be provided to regulate power to the main MCU 26, which in turn provides power to the visible light generator module 12, the magnetic field generator module 16, the infrared generator module 18, and the free electron generator module 20.

A power switch 32 may turn the energizer 10 ON/OFF by turning the power to the main MCU 26 ON/OFF. The power switch 32 may be a manual switch with a manually actuated button or an electronic switch that is wirelessly operated by another device such as a mobile device (e.g. a smart phone, a tablet, or a laptop) or a PC, for example.

The energizer 10 may further include a wireless transceiver 34 (for example, a Bluetooth transceiver chip) operatively connected to the main MCU 26 whereby the operation of the modules controlled by the main MCU 26 may be controlled by an external device such as a mobile device (e.g. a smart phone, a tablet, or a laptop) or a PC in wireless communication with the main MCU 26.

Two components 36, 38 provide two different clock frequencies for the main MCU 26. Furthermore, an audio amplifier 40 may be connected between the main MCU 26 and the visible light generator module 12, and the magnetic field generator module 16. The audio amplifier 40 can modulate the power supplied to the visible light generator module 12, and the magnetic field generator module 16 in order to increase or decrease the output thereof.

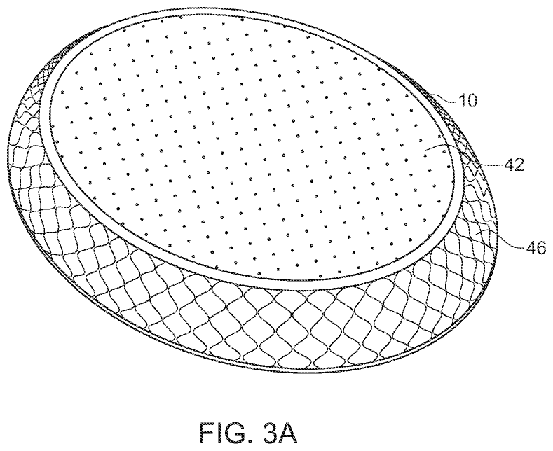

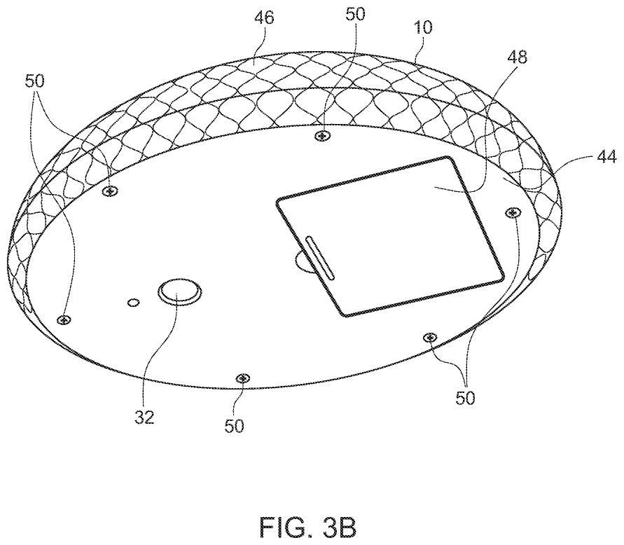

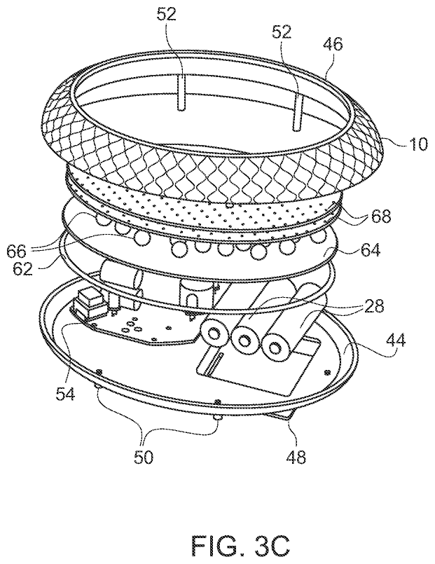

Referring to FIGS. 3A, 3B, and 3C, an energizer 10 according to one physical embodiment of the present invention is a circular (saucer-shaped) body having a flat top 42, a flat base 44, and an annular ring 46 extending between the flat top 42 and the flat base 44 and having a curved exterior surface. Preferably, the flat top 42 is circular, and the flat base 44 is also circular with a larger area than that of the flat top 42. The flat base 44 includes a removable cover 48 that provides access to a battery compartment that houses the batteries 28. The flat base 44 may be coupled to the annular ring 46 with screws 50 that are received in screw receptacles 52 located at and integrated with the interior surface of the annular ring 46. The flat base 44 and the annular ring 46 may be made of a plastic material. A manual ON/OFF switch 32 is accessible at the flat base 44. The flat base 44 may include an annular ridge defining an endless wall that makes direct contact with the lower mouth of the annular ring 46 in the assembled state.

Each module that is controlled by the main MCU 26 includes at least one output component and other control components that control the output component or components.

Referring now to FIG. 3C, a circuit board 54 carries at least the main MCU 26, and other control components that enable the generation of visible light by the visible light generator module 12, magnetic fields by the magnetic field generator module 16, infrared by the infrared generator module 18, and free electrons by the free electron generator module 20. When present, the regulator 30 may also be located on the circuit board 54. It should be noted that while the embodiment disclosed herein uses the circuit board 54 for carrying and supporting the electronic components that enable the functioning of the modules controlled by the main MCU 26, it is possible an application specific integrated circuit could be devised to perform the same functions as the components on the circuit board 54.

The circuit board 54 may be secured to the interior face of the flat base 44 with screws or glue or any other suitable means. Power may be supplied to the main MCU 26 with wires (not shown) connected to the regulator 30.

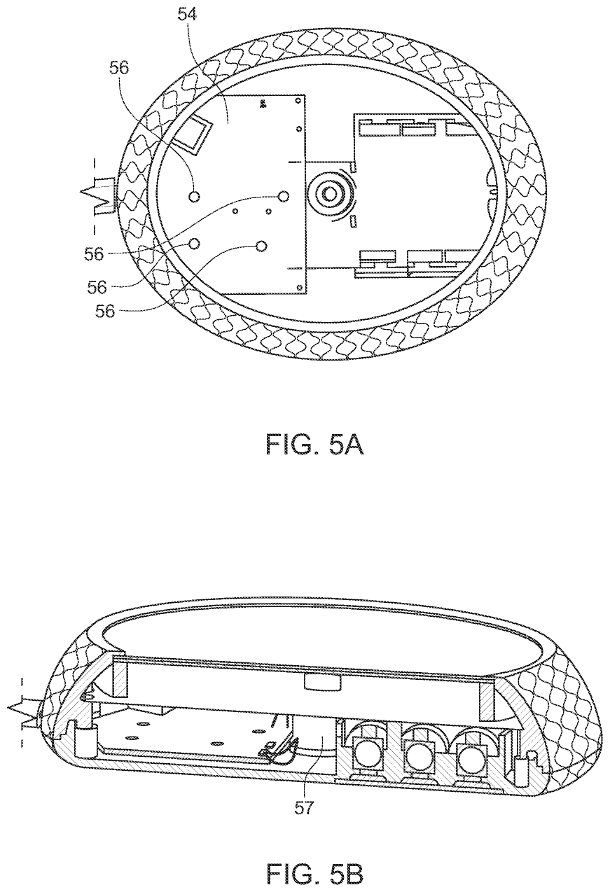

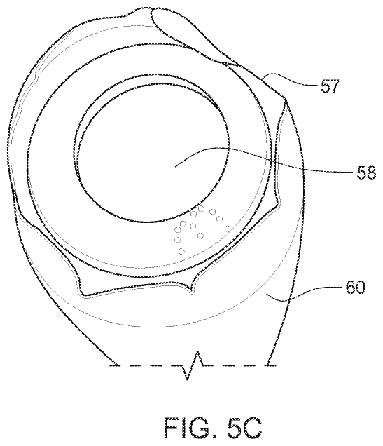

The output component of the visible light generator module 12 is an LED part(s) 56 (FIG. 5A). Each LED part 56 may be a component that emits a light at one frequency (e.g. red (650 nm), green (450 nm), blue (380 nm)) or may be a component that emits multiple frequencies of light (e.g. red (650 nm), green (450 nm), blue (380 nm), and yellow (590 nm). Irradiation with 590 nm yellow light-emitting diode attenuates oxidative stress and modulates UVB-induced change of dermal fibroblasts

The magnetic field generator module 16 includes an electromagnet 57 (FIG. 5B) that includes a permanent magnet 58 (FIG. 5C) and a coil 60 (FIG. 5C) surrounding the magnet 58 and magnetically coupled to the permanent magnet 58. The coil 60 serves as the output component of the magnetic field generator module 16. The coil 60 receives power from the main MCU 26 via the audio amplifier 40.

An IR generating coil 62 is the output component of the infrared generator module 16. The IR generating coil 62 may be a far IR (FIR) coil. FIR refers to a specific wavelength range (6000-12000 nm) within the IR spectrum.

The IR generating coil 62 and the LED part 56 may be cyclically operated (turned on and off) with a DC input at various frequencies (i.e. duty cycles). A preferred frequency is 7.83 Hz, which is known as the Schumann Resonance. Stimulus delivered at the Schumann Resonance can alleviate insomnia, which is mostly caused by toxin accumulation (including hormones like cortisol inside the brain). The IR generating coil 62 and the LED component 56 may be operated at other harmonic frequencies, including 14.07 Hz, 20.25 Hz, 26.41 Hz, and 32.45 Hz to address the cause(s) of dementia. The operation frequency range of the IR generating coil 62 and the LED part 56 may be 5-50 Hz. The exact frequency can be set by the user, for example, through an app on a mobile device that is wirelessly connected to a CDD according to the present invention.

The free electron generator module 20 is a DC power generator circuit, with a metallic body (e.g. the exterior output plate 68 with the surface 42 discussed below) that generates a static electric field of 850V DC. The 850V static field can reverse the downside effects of the brain leading to dementia/Parkinson's disease.

The energy field integrator 14 has a plurality of gemstones 66 cut to specific dimensions to generate torsion fields aided in part by the magnetic field generated by the magnetic field generator module 16. The energy field integrator 14 includes a base panel 64, and output plates 68. The plurality of gemstones 66 reside between the base panel 64 and the output plates 68. The base plate 64, and the output plates 68 are preferably circular bodies. The topmost output plate 68 defines the flat top 42 of the energizer 10. The base panel 64 is preferably at least partially transparent to the output of the modules controlled by the main MCU 26. The output plates 68 are preferably made from stainless steel and are perforated to create wave diffraction patterns. The number, the spacing, and the size of the perforations are not critical, but more perforations lead to better diffraction.

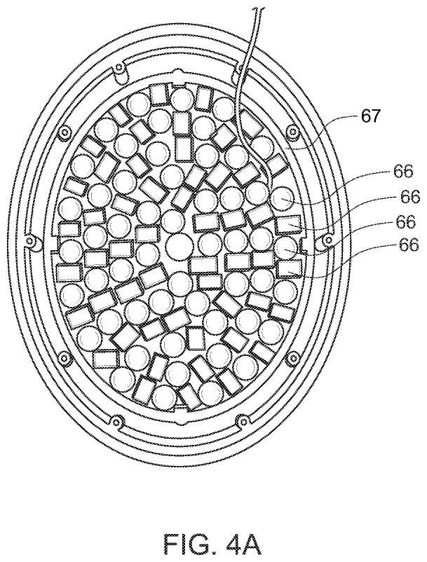

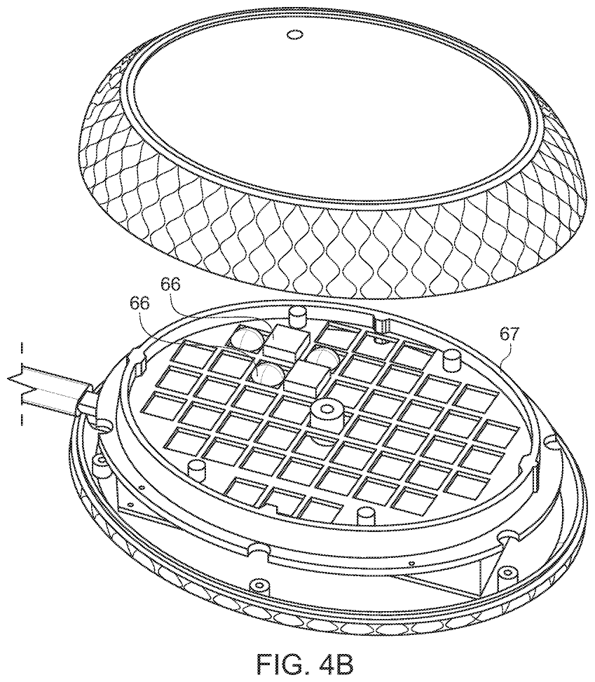

The gemstones 66 of the energy field integrator 14 serve as the output components thereof to generate torsion fields. It is known that the shape of a torsion field can be controlled by the proper selection of the geometry of the object that generates the torsion field. Some of the gemstones 66 may be finely polished spherical gemstones and others may be finely polished rectangular cubes each having a width to height ratio corresponding to the height to base perimeter ratio of the great pyramid. Finely polished as used herein means mirror bright finish with clear reflection and no haze, with all surface defects removed, which may be attained by buffing and then color buffing to achieve a mirror finish. The gemstones 66 are preferably located in a tray 67 (FIG. 4A) having receptacles (FIG. 4B), each for receiving a respective gemstone 66.

The base plate 64 may be replaced with the tray 67. The tray 67 is also at least partially transparent to the outputs of the modules of the energizer 10.

The gemstones 66 are arranged in an ordered array to integrate the light energy, the free electrons (at a potential difference of 850V to generate an electron field), and the magnetic field to obtain an integrated energy torsion field that rotates and spreads out in spherical form covering an effective space of 1M×1M×1M. Thus, when the energizer is placed under a pillow, a person's head could reside within the integrated energy torsion field. The integrated energy torsion field can induce a state of deep sleep (with anti-inflammatory effect on the affected brain cells or neural networks) and boost the cerebral spinal fluid (CSF) flow inside the skull (resulting in the removal of toxin and metabolic wastes in the brain cells or neural networks). The energy field integrator 14 receives the output of the output components of the modules controlled by the main MCU 26 through the base panel 64, integrates the outputs so received, and outputs the integrated outputs through the output plates 68.

For the sake of clarity, the control components of the modules are not shown in the figures. It is submitted that the control components that control the output component(s) of each module and their manner of connection to the output component(s) are known and need not be described in detail.

In one embodiment, an app residing on a mobile device such as a smart phone may be used to control/and adjust the output of the modules including the frequency, the light photon intensity, and the electrical power output.

An energizer according to the present invention may be used all the time (24/7). It can be made small enough to be carried in one's pocket or under a pillow. It can be operated to strengthen the body's energy field in order to enhance brain toxin removal, facilitate hormonal balance, enhance cranial blood circulation, and enhance brain cell and neural network repair.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. An energizer, comprising:
a plurality of energy producing modules each configured to emit a respective energy output of a type different from energy outputs of other modules; and
an energy field integrator arranged to receive the energy outputs of the energy producing modules, to integrate the received energy outputs, and to output an integrated output, the energy field integrator comprising a plurality of gemstones arranged in an ordered array.

2. The energizer of claim 1, wherein the integrated output is a torsion field.

3. The energizer of claim 2, wherein the torsion field is spherical.

4. The energizer of claim 2, wherein the plurality of gemstones includes spherical gemstones and cubical gemstones.

5. The energizer of claim 4, wherein the energy field integrator includes a base panel, and at least one output plate, wherein the base panel is at least partially transparent to the outputs of the modules, and wherein the plurality of gemstones resides between the base panel and the at least one output plate.

6. The energizer of claim 5, wherein the at least one output plate includes a plurality of spaced perforations arranged to create wave diffraction patterns.

7. The energizer of claim 4, wherein the energy field integrator further includes a tray having a plurality of receptacles, and the plurality of gemstones are received in the receptacles, and wherein the tray is at least partially transparent to the outputs of the modules.

8. The energizer of claim 1, wherein the modules include a visible light generator module, a magnetic field generator module, an infrared generator module, and a free electron generator module.

9. The energizer of claim 8, wherein the modules include a body infrared signal generator module.

10. The energizer of claim 8, further comprising a microcontroller that controls the modules.

11. The energizer of claim 8, wherein the visible light generator module comprises a plurality of LED components serving as output components thereof.

12. The energizer of claim 11, wherein the LED components include LEDs that emit light with wavelengths of 650 nm, 525 nm, 380 nm, and 590 nm.

13. The energizer of claim 11, wherein the infrared generator module comprises an infrared (IR) generating coil serving as an output component of the infrared generator module emitting infrared in the range 6000 nm to 12000 nm.

14. The energizer of claim 8, wherein the magnetic field generator module comprises an electromagnet serving as an output component thereof.

15. The energizer of claim 14, wherein the magnetic field generator module generates a magnetic field in the range 0.1 to 80 microteslas.

16. The energizer of claim 8, wherein the free electron generator module is a DC power generator.

17. The energizer of claim 8, wherein the infrared generator module and the visible light generator module are configured to be cyclically operated at a frequency set by a user.

18. The energizer of claim 17, wherein the frequency may be 7.83 Hz or a harmonic thereof.

19. The energizer of claim 1, wherein output of the modules are adjustable in response to user instructions.

20. The energizer of claim 19, wherein user instructions are received from a user-operable mobile device.
